(19) United States
(12) Patent Application Publication
(10) Pub. No.: US 2012/0220783 A1
(43) Pub. Date: Aug. 30, 2012

(54) SALTS OF SUNITINIB

(75) Inventors: Sudhir Singh Sanwal, Kangra (IN); Saridi Madhava Dileep Kumar, Gurgaon (IN); Swargam Sathyanarayana, Karim Nagar (IN); Rajesh Kumar Thaper, Jammu (IN); Mohan Prasad, Gurgaon (IN)

(73) Assignee: RANBAXY LABORATORIES LIMITED, New Delhi, Delhi (IN)

(21) Appl. No.: 13/496,559

(22) PCT Filed: Sep. 16, 2010
(86) PCT No.: PCT/IB2010/054196

§ 371 Date: May 9, 2012

(30) Foreign Application Priority Data
Sep. 16, 2009 (IN) 1932/DEL/2009

(51) Int. Cl. C07D 403/06

(52) U.S. Cl. 548/468

(57) ABSTRACT

The present invention relates to salts of sunitinib and their preparation.

### FIELD OF THE INVENTION

The present invention relates to salts of sunitinib and processes for their preparation.

### BACKGROUND OF THE INVENTION

Sunitinib is chemically described as N-[2-(diethylamino)ethyl]-5-[(Z)-(5 -fluoro-1,2-dihydro-2-oxo-3H-indo1-3-ylidine)methyl]-2,4-dimethyl-1H-pyrrole-3-carboxamide as represented by Formula I.

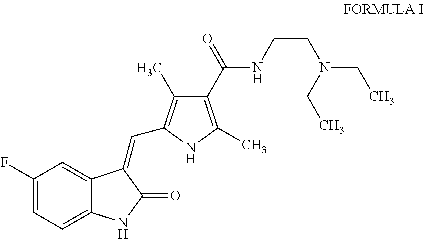

Sunitinib is an oral multi-kinase inhibitor and is used in the treatment of gastrointestinal stromal tumor and advanced renal cell carcinoma. Sunitinib is commercially available as the L-malate salt, which is described chemically as butanedioic acid, hydroxy-, (2S)-, compound with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1).

U.S. Application Publication Nos. 2003/0069298 and 2007/0191458 describe the preparation of crystalline Forms I and II of the L-malic acid salt of sunitinib. WO 2009/067686 describes processes for preparing crystalline forms of racemic sunitinib malate, sunitinib hemi-L-malate and compositions containing sunitinib base and L- or racemic malic acid. WO 2009/104021 describes processes for preparing crystalline Forms III and IV of sunitinib L-malate. WO 2009/104021 states that Form II of sunitinib L-malate is hygroscopic, thermodynamically unstable and appears to readily convert to Form I.

The present inventors have prepared salts of sunitinib with achiral acids. The salts of the present invention are easy to prepare and isolate in solid forms particularly in crystalline forms, stable and efficient to prepare pharmaceutical dosage forms.

### SUMMARY OF THE INVENTION

In one general aspect, the present invention provides for a salt of sunitinib with an achiral acid. Embodiments of this aspect may include one or more of the following features. For example, the achiral acid is an organic or inorganic acid. Suitable achiral acid include citric acid, p-toluenesulfonic acid, sulfuric acid, acetic acid, methanesulfonic acid, maleic acid, fumaric acid, ethanesulfonic acid or succinic acid. The salt of sunitinib with an achiral acid is in a solid form. The solid form may be a crystalline form.

In another general aspect, the present invention provides for a crystalline salt of sunitinib with citric acid. Embodiments of this aspect may include one or more of the following features. For example, the crystalline salt of sunitinib with citric acid includes an XRPD pattern comprising any five of interplanar spacing (d) values selected from the group consisting of 15.72, 10.50, 9.39, 8.71, 7.19, 7.00, 6.50, 6.30, 5.70, 5.44, 5.38, 5.10, 4.72, 4.36, 4.21, 3.57, 3.50, 3.40, 3.28 and 3.14 (Å).

In another general aspect, the present invention provides for a crystalline salt of sunitinib with p-toluenesulfonic acid. Embodiments of this aspect may include one or more of the following features. For example, the crystalline salt of sunitinib with p-toluenesulfonic acid includes an XRPD pattern comprising any five of interplanar spacing (d) values selected from the group consisting of 18.51, 9.25, 8.05, 7.45, 7.04, 6.55, 6.17, 5.72, 5.38, 5.14, 4.96, 4.90, 4.84, 4.70, 4.59, 4.52, 4.33, 4.24, 4.15, 4.03, 3.91, 3.79, 3.70, 3.65, 3.54, 3.46, 3.36, 3.30, 3.20 and 3.11 (Å).

In yet another general aspect, the present invention provides for a crystalline salt of sunitinib with sulfuric acid. Embodiments of this aspect may include one or more of the following features. For example, the crystalline salt of sunitinib with sulfuric acid includes an XRPD pattern comprising any five of interplanar spacing (d) values selected from the group consisting of 15.34, 13.23, 10.08, 9.53, 8.76, 7.66, 7.48, 7.04, 6.62, 6.54, 6.17, 6.06, 5.74, 5.49, 5.27, 5.10, 4.88, 4.82, 4.72, 4.50, 4.41, 4.31, 4.19, 4.07, 4.00, 3.97, 3.93, 3.83, 3.70, 3.65, 3.60, 3.54, 3.47, 3.44, 3.40, 3.27, 3.19 and 2.92 (Å).

In another general aspect, the present invention provides for a crystalline salt of sunitinib with acetic acid. Embodiments of this aspect may include one or more of the following features. For example, the crystalline salt of sunitinib with acetic acid includes an XRPD pattern comprising any five of interplanar spacing (d) values selected from the group consisting of 23.47, 14.60, 12.35, 11.68, 9.74, 8.06, 7.79, 6.70, 6.53, 6.09, 5.59, 5.41, 5.16, 4.94, 4.86, 4.68, 4.40, 4.15, 4.04, 4.00, 3.80, 3.65, 3.60, 3.54, 3.47, 3.41, 3.32, 3.28 and 3.04 (Å).

In another general aspect, the present invention provides for a crystalline salt of sunitinib with methanesulfonic acid. Embodiments of this aspect may include one or more of the following features. For example, the crystalline salt of sunitinib with methanesulfonic acid includes an XRPD pattern comprising any five of interplanar spacing (d) values selected from the group consisting of 15.18, 9.46, 8.58, 7.57, 7.03, 6.61, 6.36, 6.18, 5.78, 5.53, 5.28, 5.10, 5.04, 4.77, 4.72, 4.58, 4.34, 4.28, 4.21, 4.06, 3.91, 3.83, 3.78, 3.71, 3.68, 3.64, 3.58, 3.51, 3.43, 3.35, 3.26, 3.23, 3.15 and 3.00 (Å).

In another general aspect, the present invention provides for a process for the preparation of a salt of sunitinib with an achiral acid. The process includes treating sunitinib with an achiral acid. Embodiments of this aspect may include one or more of the following features. For example, the treatment with the achiral acid is carried out in the presence of a solvent. Suitable solvents include organic solvents. The organic solvent may be a water-miscible organic solvent. The water-miscible organic solvent may be a C1-3 alkanol.

The achiral acid for use in the process includes citric acid, p-toluenesulfonic acid, sulfuric acid, acetic acid, methanesulfonic acid, maleic acid, fumaric acid, ethanesulfonic acid or succinic acid. DR

### BRIEF DESCRIPTION OF THE DRAWINGS

FIG. 1 depicts the X-ray powder diffraction pattern (XRPD) of the salt of sunitinib with citric acid.

FIG. 1A provides the XRPD values corresponding to FIG. 1.

FIG. 2 depicts the X-ray powder diffraction pattern (XRPD) of the salt of sunitinib with p-toluenesulfonic acid.

FIG. 2A provides the XRPD values corresponding to FIG. 2.

FIG. 3 depicts the X-ray powder diffraction pattern (XRPD) of the salt of sunitinib with sulfuric acid.

FIG. 3A provides the XRPD values corresponding to FIG. 3.

FIG. 4 depicts the X-ray powder diffraction pattern (XRPD) of the salt of sunitinib with acetic acid.

FIG. 4A provides the XRPD values corresponding to FIG. 4.

FIG. 5 depicts the X-ray powder diffraction pattern (XRPD) of the salt of sunitinib with methanesulfonic acid.

FIG. 5A provides the XRPD values corresponding to FIG. 5.

### DETAILED DESCRIPTION OF THE INVENTION

A first aspect of the present invention provides a salt of sunitinib with an achiral acid. The achiral acid is an organic or inorganic acid. The achiral acid may be, for example, citric acid, p-toluenesulfonic acid, sulfuric acid, acetic acid, methanesulfonic acid, maleic acid, fumaric acid, ethanesulfonic acid or succinic acid. The salt of sunitinib with an achiral acid may be in a solid form, such as, a crystalline form.

The term “salt of sunitinib” includes a combination of sunitinib and an acid in any ratio between about 1:0.25 and about 1:5. The term “achiral acid” refers to an acid that does not have a chiral center.

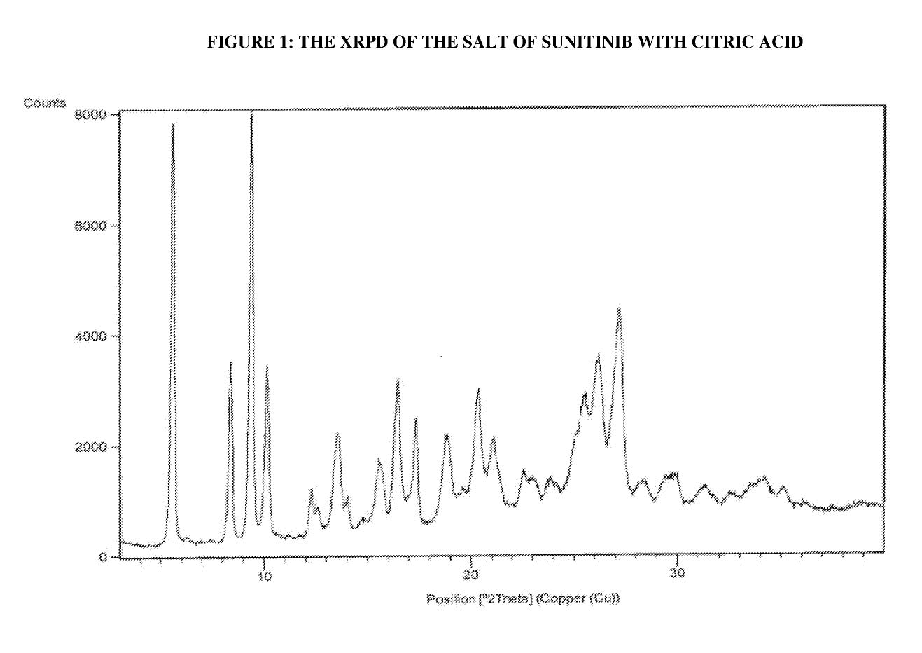

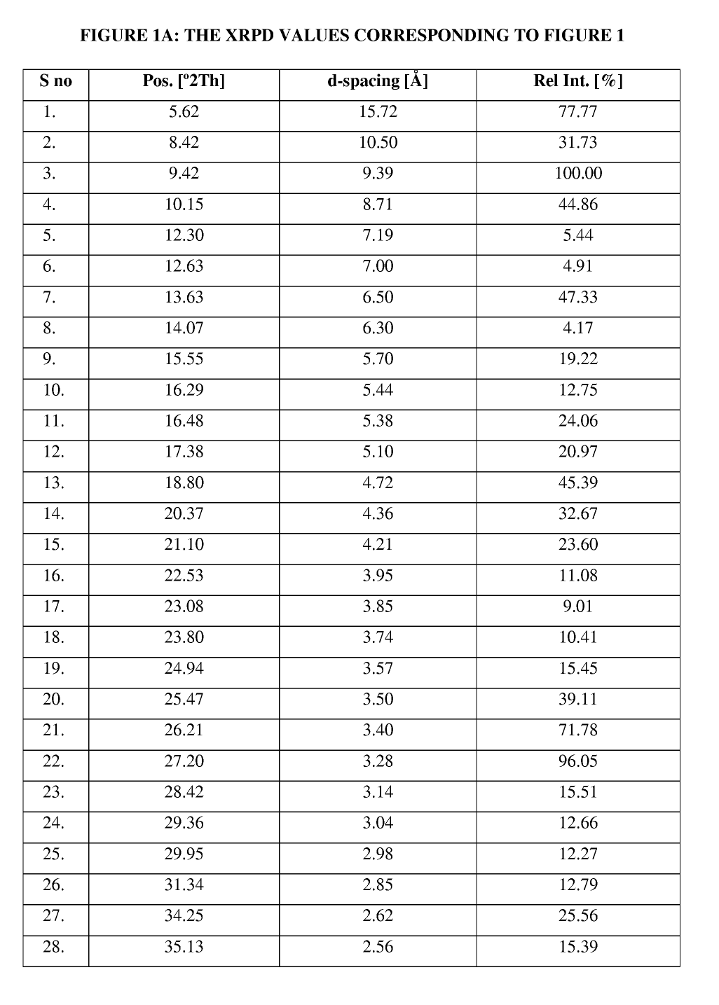

A second aspect of the present invention provides a crystalline salt of sunitinib with citric acid. The crystalline salt of sunitinib with citric acid may be characterized by an XRPD pattern, which includes the following interplanar spacing (d) values: 15.72, 9.39, 6.50, 4.72, and 3.28 (Å). The XRPD may also include the following interplanar spacing (d) values: 10.50, 8.71, 7.19, 7.00, 6.30, 5.70, 5.44, 5.38, 5.10, 4.26, 4.21, 3.57, 3.50, 3.40 and 3.14 (Å). The crystalline salt of sunitinib with citric acid may be further characterized by substantially the same XRPD pattern as depicted in FIG. 1.

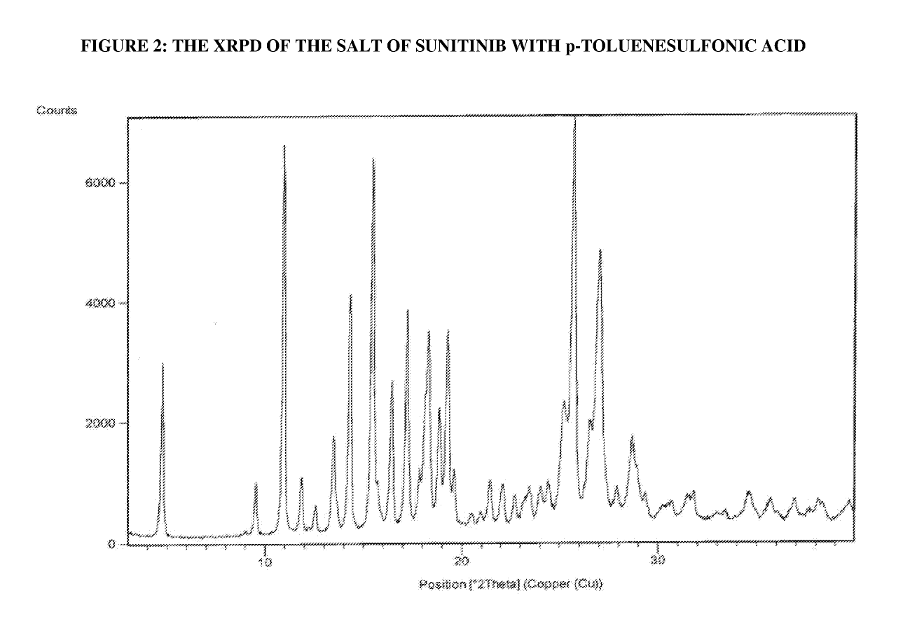

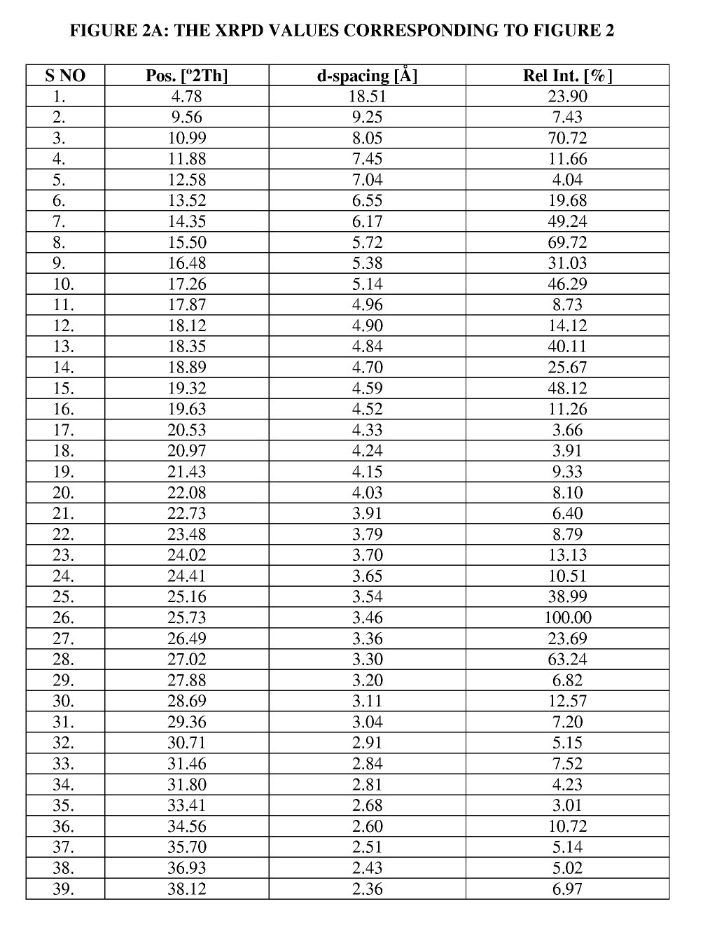

A third aspect of the present invention provides a crystalline salt of sunitinib with p-toluenesulfonic acid. The crystalline salt of sunitinib with p-toluenesulfonic acid may be characterized by an XRPD pattern, which includes the following interplanar spacing (d) values: 8.05, 6.17, 5.72, 4.59, 3.46, and 3.30 (Å). The XRPD may also include the following interplanar spacing (d) values: 18.51, 9.25, 7.45, 7.04, 6.55, 5.38, 5.14, 4.96, 4.90, 4.84, 4.70, 4.52, 4.33, 4.24, 4.15, 4.03, 3.91, 3.79, 3.70, 3.65, 3.54, 3.36, 3.20 and 3.11 (Å). The crystalline salt of sunitinib with p-toluenesulfonic acid may be further characterized by substantially the same XRPD pattern as depicted in FIG. 2.

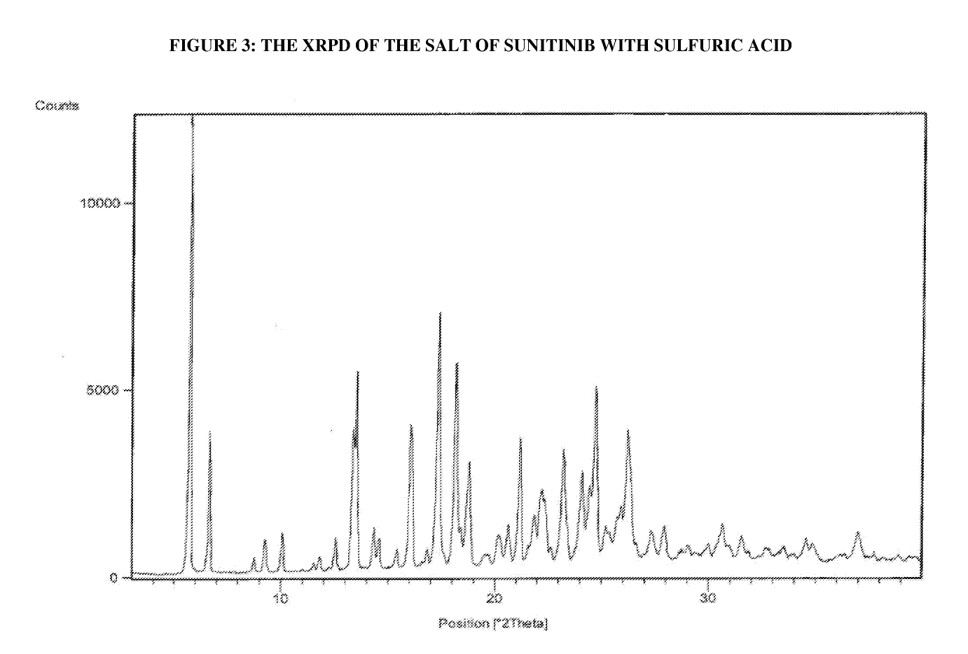

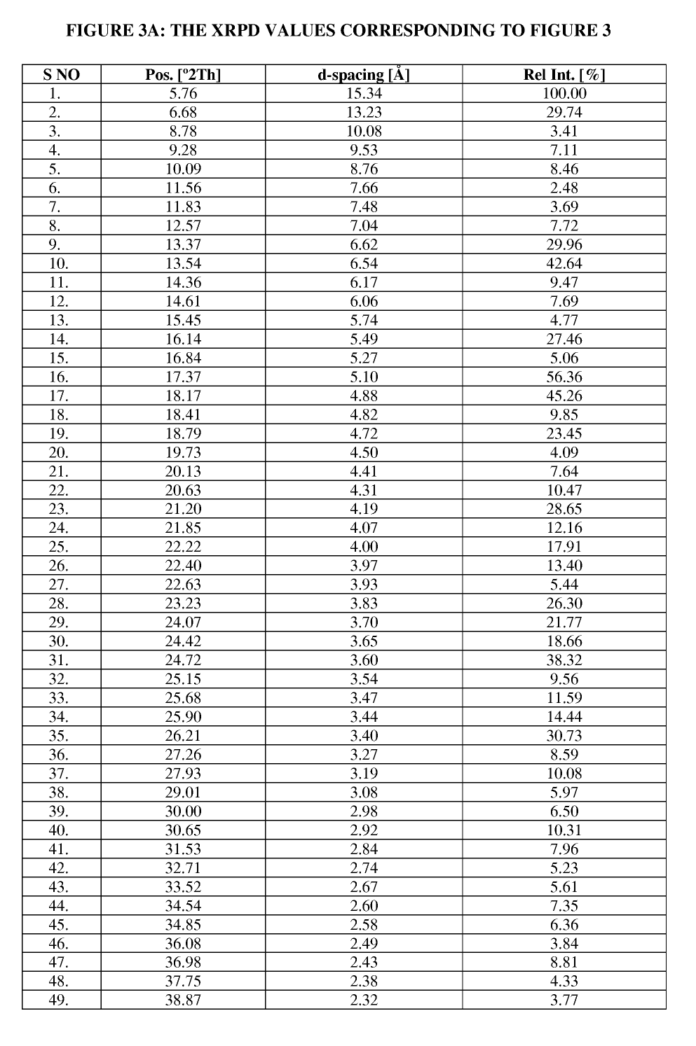

A fourth aspect of the present invention provides a crystalline salt of sunitinib with sulfuric acid. The crystalline salt of sunitinib with sulfuric acid may be characterized by an XRPD pattern, which includes the following interplanar spacing (d) values: 15.34, 6.54, 5.10, 4.88, 3.60, and 3.40 (Å). The XRPD may also include the following interplanar spading (d) values: 13.23, 10.08, 9.53, 8.76, 7.66, 7.48, 7.04, 6.62, 6.17, 6.06, 5.74, 5.49, 5.27, 4.82, 4.72, 4.50, 4.41, 4.31, 4.19, 4.07, 4.00, 3.97, 3.93, 3.83, 3.70, 3.65, 3.54, 3.47, 3.44, 3.27, 3.19 and 2.92 (Å). The crystalline salt of sunitinib with sulfuric acid may be further characterized by substantially the same XRPD pattern as depicted in FIG. 3.

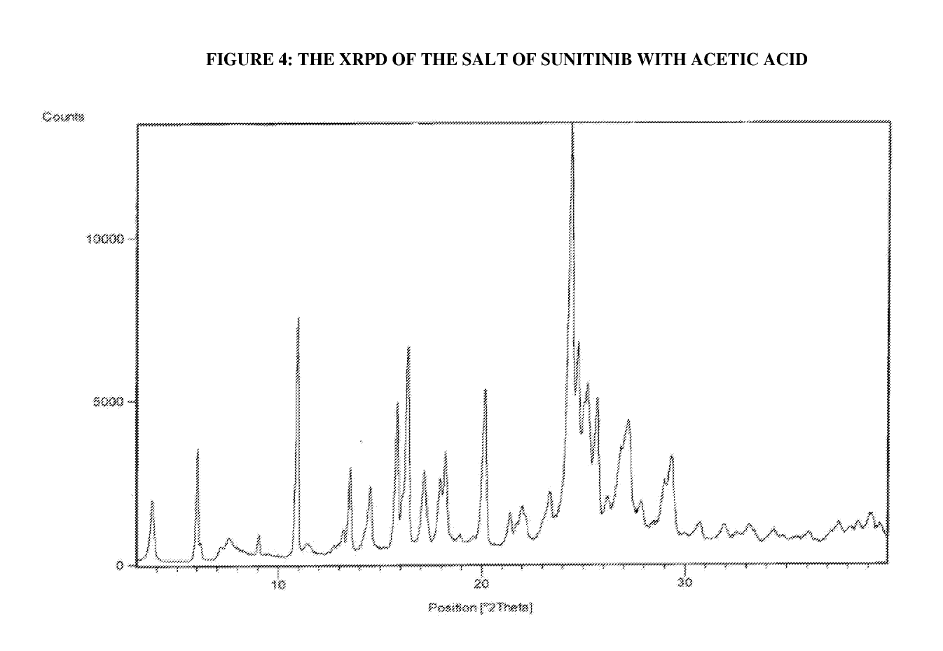

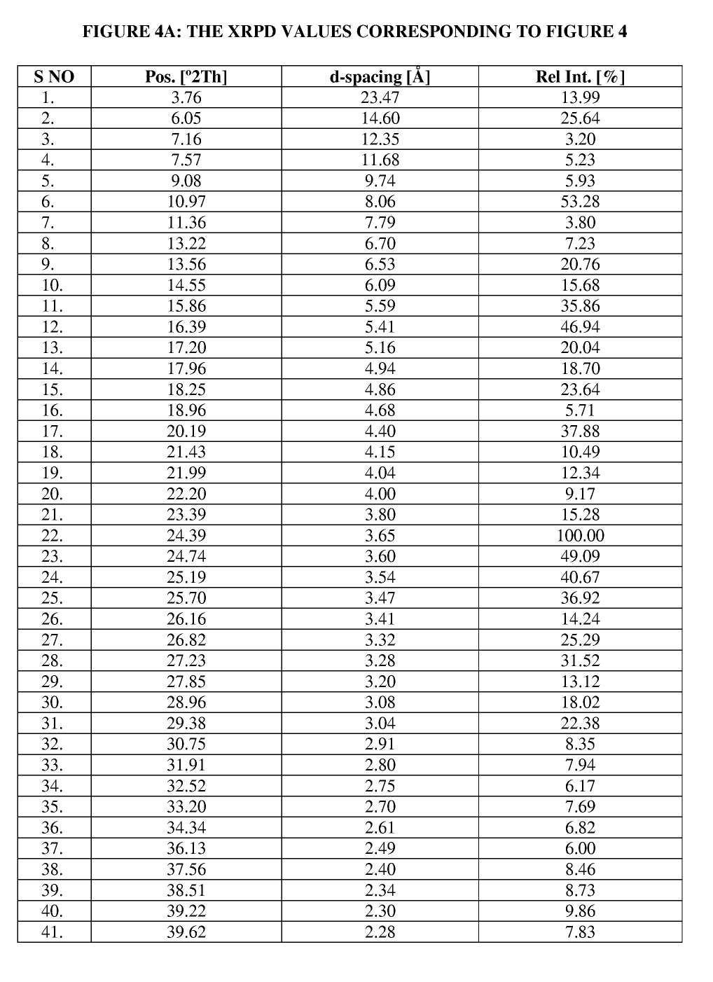

A fifth aspect of the present invention provides a crystalline salt of sunitinib with acetic acid. The crystalline salt of sunitinib with acetic acid may be characterized by an XRPD pattern, which includes the following interplanar spacing (d) values: 8.06, 5.41, 4.40, 3.65, 3.60, and 3.54 (Å). The XRPD may also include the following interplanar spacing (d) values: 23.47, 14.60, 12.35, 11.68, 9.74, 7.79, 6.70, 6.53, 6.09, 5.59, 5.16, 4.94, 4.86, 4.68, 4.15, 4.04, 4.00, 3.80, 3.47, 3.41, 3.32, 3.28 and 3.04 (Å). The crystalline salt of sunitinib with acetic acid may be further characterized by substantially the same XRPD pattern as depicted in FIG. 4.

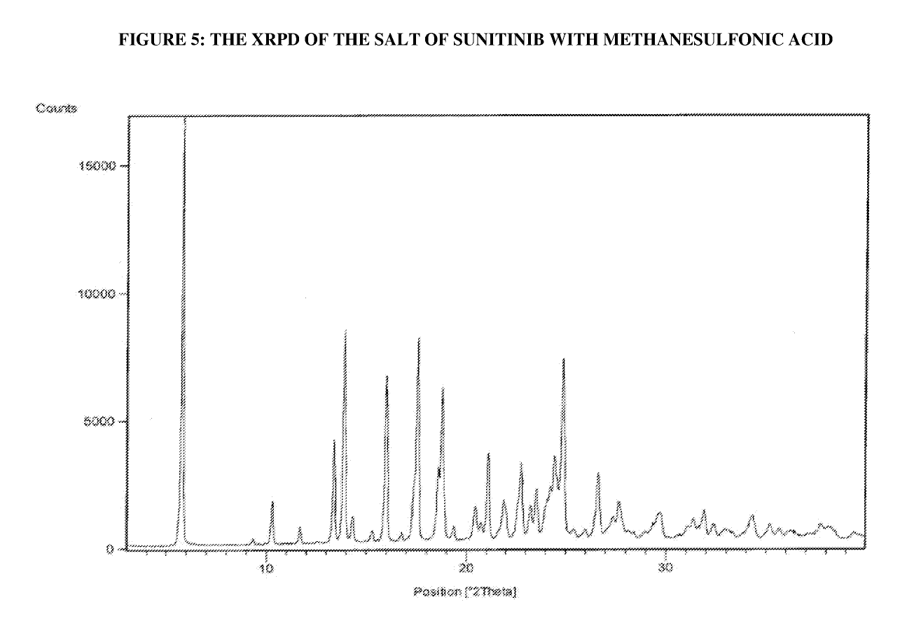

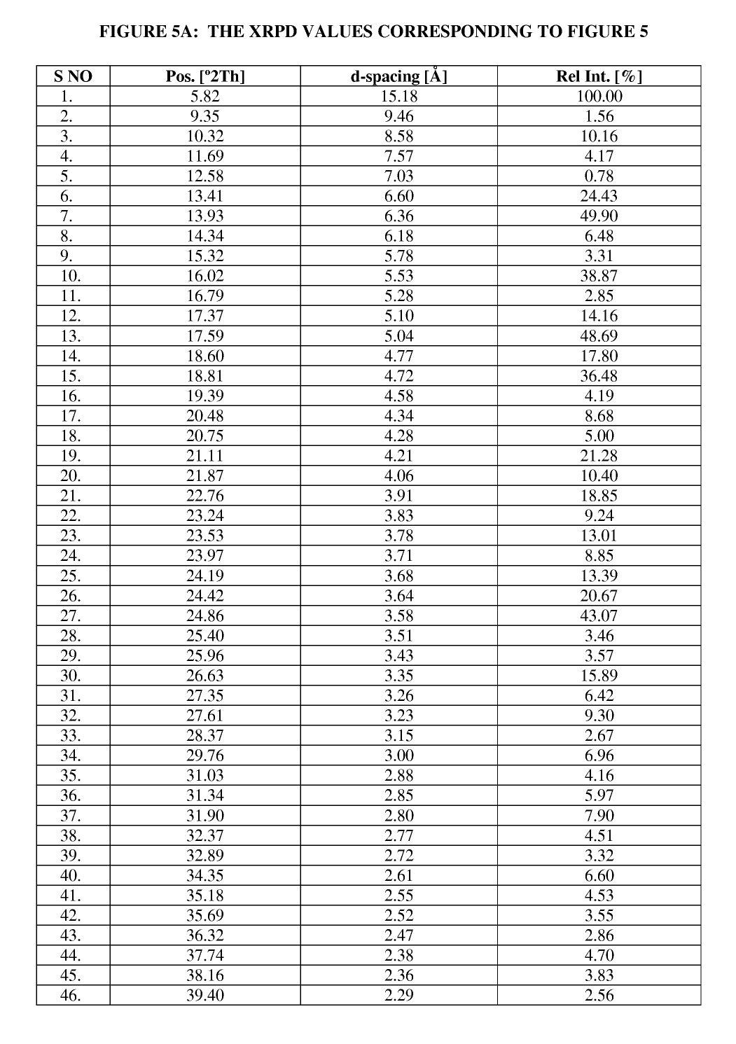

A sixth aspect of the present invention provides a crystalline salt of sunitinib with methanesulfonic acid. The crystalline salt of sunitinib with methanesulfonic acid may be characterized by an XRPD pattern, which includes the following interplanar spacing (d) values: 15.18, 6.36, 5.53, 5.04, 4.72, 3.64, and 3.58 (Å). The XRPD may also include the following interplanar spacing (d) values: 9.46, 8.58, 7.57, 7.03, 6.61, 6.18, 5.78, 5.28, 5.10, 4.77, 4.58, 4.34, 4.28, 4.21, 4.06, 3.91, 3.83, 3.78, 3.71, 3.68, 3.51, 3.43, 3.35, 3.26, 3.23, 3.15 and 3.00 (Å). The crystalline salt of sunitinib with methanesulfonic acid may be further characterized by substantially the same XRPD pattern as depicted in FIG. 5.

A seventh aspect of the present invention provides a process for the preparation of a salt of sunitinib with an achiral acid, wherein the process includes a step of treating sunitinib with an achiral acid.

The starting sunitinib may be prepared according to the method provided in U.S. Pat. No. 6,573,293. The sunitinib is treated with an achiral acid, for example, citric acid, p-toluenesulfonic acid, sulfuric acid, acetic acid, methanesulfonic acid, maleic acid, fumaric acid, ethanesulfonic acid or succinic acid. The treatment with the achiral acid may be carried out in the presence of a solvent, such as, an organic solvent. The organic solvent may be a water-miscible organic solvent, for example, a C₁₋₃alkanol. The treatment may be carried out at a temperature of about 15° C. to about 65° C., for example, from about 20° C. to about 60° C. The formation of the salt of sunitinib with the achiral acid may be facilitated by stirring the reaction mixture for about 5 minutes to about 50 hours. The salt of sunitinib with the achiral acid may be isolated by filtration, decantation, solvent precipitation, trituration with a hydrocarbon, for example, n-hexane, evaporation, distillation or a combination thereof.

An eighth aspect of the present invention provides a pharmaceutical composition comprising a salt of sunitinib with an achiral acid and a carrier. The achiral acid may be, for example, citric acid, p-toluenesulfonic acid, sulfuric acid, acetic acid, methanesulfonic acid, maleic acid, fumaric acid, ethanesulfonic acid or succinic acid. The salt of sunitinib with an achiral acid may be in a solid form, for example, a crystalline form.

Another aspect of the present invention provides a pharmaceutical composition comprising a crystalline salt of sunitinib with citric acid, sunitinib with p-toluenesultonic acid, sunitinib with sulfuric acid, sunitinib with acetic acid, or sunitinib with methanesulfonic acid and a pharmaceutical acceptable carrier.

A final aspect of the present invention provides a method of treating or preventing a protein kinase related disorder, which includes administering to a patient in need thereof a therapeutically effective amount of a salt of sunitinib with an achiral acid. The achiral acid may be, for example, citric acid, p-toluenesulfonic acid, sulfuric acid, acetic acid, methanesulfonic acid, maleic acid, fumaric acid, ethanesulfonic acid or succinic acid.

XRPD of the samples were determined by using Panalytical X'Pert Pro X-Ray Powder Diffractometer in the range 3-40 degree 2 theta and under tube voltage and current of 45 Kv and 40 mA respectively. Copper radiation of wavelength 1.54 angstrom and Xceletor detector was used.

While the present invention has been described in terms of its specific embodiments, certain modifications and equivalents will be apparent to those skilled in the art and are intended to be included within the scope of the present invention.

### EXAMPLES

### Example 1

### Preparation of Sunitinib

### Step-1

### Preparation of N-[2-(diethyl amino) ethyl-5-formyl-2,4 dimethyl-1H pyrrole-3-carboxamide

A mixture of 5-formyl-2,4-dimethyl-1H-pyrrole-3-carboxylic acid (100 g) in dimethylformamide (900 ml) was stirred for 15 minutes. 1-(3-Dimethylaminopropyl)-3-ethylcarbodiimide hydrochloride (171.8 g), 1-hydroxybenzotriazole (121.2 g) and triethylamine (169.4 g) were added to the reaction mixture and stirred at 20° C. to 25° C. for 10 minutes. N,N-Diethylethylenediamine (104.2 g) was slowly added to the reaction mixture and stirred at 20° C. to 25° C. for 20 hours. The reaction mixture was diluted with water (350 ml), brine (250 ml) and saturated sodium bicarbonate solution (350 ml) followed by the addition of 10% v/v methanol in dichloromethane (1500 ml). The reaction mixture was stirred for 30 minutes and allowed to settle for 30 minutes. The organic layer was separated and washed with a saturated sodium bicarbonate solution (1500 ml). The organic layer was separated, dried over sodium sulfate and concentrated to obtain a residue. Toluene (300 ml) was added to the residue and evaporated to dryness. Ethyl acetate (600 ml) was added to the residue and washed with brine (400 ml). The organic layer was separated, dried over sodium sulfate and concentrated under vacuum to obtain a sticky solid. The solid so obtained was triturated with hexane:diethyl ether (3:1; 500 ml), stirred for 15 minutes, filtered under vacuum and dried under vacuum at 55° C. for 16 hours to obtain the title compound.

Yield: 44 g

### Step-2:

### Preparation of Sunitinib

A mixture of N-[2-(Diethylamino)ethyl-5-formyl-2,4-dimethyl-1H-pyrrole-3-carboxamide (8.0 g) in ethanol (80 mL) was stirred. 5-Fluoro-1,3-dihydroindol-2-one (4.5 g) and pyrrolidine (0.123 ml) were added to the reaction mixture and stirred at 78° C. (internal temperature) for 1 hour. After 1 hour at 78° C., additional ethanol (20 ml) was added and the stirring was continued for an additional 2 hours. The reaction mixture was cooled to 20° C. to 25° C. and filtered under vacuum. The solid was stirred at 72° C. (internal temperature) in ethanol (80 ml) for 30 minutes. The reaction mixture was cooled to 20° C. to 25° C., filtered under vacuum, washed with ethanol (2×50 ml) and dried under vacuum at 55° C. to 60° C. for 42 hours to obtain the title compound.

Yield: 8.2 g

### Example 2

### Preparation of a Salt of Sunitinib with Citric Acid

A mixture of sunitinib (5 g) in methanol (100 mL) was stirred for 30 minutes at 40° C. Citric acid monohydrate (2.76 g) was added to the reaction mixture and stirred at 55° C. for 1 hour. The reaction mixture was cooled to 20° C. to 25° C. and stirred for 2 hours. Methanol was removed from the reaction mixture under reduced pressure to obtain a solid. The solid was dried under vacuum at 50° C. for 16 hours to obtain the title salt having an XRPD pattern as depicted in FIG. 1.

Yield: 7.7 g

### Example 3

### Preparation of a Salt of Sunitinib with p-Toluenesulfonic Acid

A mixture of sunitinib (4 g) in methanol (35 ml) was stirred for 10 minutes at 20° C. to 25° C. p-Toluenesulfonic acid (2.0 g) solution in methanol (13 ml) was added slowly into the reaction mixture and stirred at 20° C. to 25° C. for 4 hours. The reaction mixture was filtered under vacuum and dried under vacuum at 55° C. for 18 hours to obtain the title salt having an XRPD pattern as depicted in FIG. 2.

Yield: 5.5 g

### Example 4

### Preparation of a Salt of Sunitinib with Sulfuric Acid

A mixture of sunitinib (4 g) in methanol (40 ml) was stirred for 10 minutes at 20° C. to 25° C. Sulfuric acid (1.03 g) dissolved in methanol (10 ml) was slowly added into the reaction mixture and stirred at 20° C. to 25° C. for 10 minutes. The reaction mixture was heated at 60° C. (external temperature) for about 3 hours and cooled to 20° C. to 25° C. The solid was filtered under vacuum at 20° C. to 25° C. and dried under vacuum at 55° C. for 16 hours to obtain the title salt having an XRPD pattern as depicted in FIG. 3.

Yield: 4.8 g

### Example 5

### Preparation of a Salt of Sunitinib with Acetic Acid

A mixture of sunitinib (4 g) in methanol (40 ml) was stirred for 10 minutes at 20° C. to 25° C. Acetic acid (0.632 g) dissolved in methanol (10 ml) was added slowly into the reaction mixture and stirred at 20° C. to 25° C. for 10 minutes. The reaction mixture was heated at 60° C. (external temperature) for 3 hours, cooled to 20° C. to 25° C., concentrated under vacuum and dried for 1 hour. The residue was triturated with n-hexane (100 ml) and stirred at 20° C. to 25° C. for 15 hours. The solid was filtered under vacuum and dried under vacuum at 60° C. for 24 hours to obtain the title salt having an XRPD pattern as depicted in FIG. 4.

Yield: 4.3 g

### Example 6

### Preparation of a Salt of Sunitinib with Methanesulfonic Acid

A mixture of sunitinib (4 g) in methanol (40 ml) was stirred for 15 minutes at 20° C. to 25° C. Methanesulfonic acid (1.01 g) dissolved in methanol (20 ml) was added slowly into the reaction mixture and stirred at 20° C. to 25° C. for 15 to 20 minutes to obtain a precipitate. The stirring was continued at 20° C. to 25° C. for an additional 2 hours. The solid was filtered under vacuum and dried under vacuum for 20 hours to obtain the title solid having an XRPD pattern as depicted in FIG. 5.

Yield: 4.2 g

1. (canceled)

2. (canceled)

3. A salt of sunitinib with sulfuric acid, acetic acid, methanesulfonic acid, ethanesulfonic acid or succinic acid.

4. A salt of sunitinib according to claim 3, wherein the salt of sunitinib with sulfuric acid, acetic acid, methanesulfonic acid, ethanesulfonic acid or succinic acid is in a solid form.

5. A salt of sunitinib according to claim 4, wherein the solid form comprises a crystalline form.

6. A crystalline salt of sunitinib with citric acid.

7. A crystalline salt of sunitinib with citric acid having an XRPD pattern comprising any five of interplanar spacing (d) values selected from the group consisting of 15.72, 10.50, 9.39, 8.71, 7.19, 7.00, 6.50, 6.30, 5.70, 5.44, 5.38, 5.10, 4.72, 4.36, 4.21, 3.57, 3.50, 3.40, 3.28 and 3.14 (Å).

8. A crystalline salt of sunitinib with p-toluenesulfonic acid.

9. A crystalline salt of sunitinib with p-toluenesulfonic acid having an XRPD pattern comprising any five of interplanar spacing (d) values selected from the group consisting of 18.51, 9.25, 8.05, 7.45, 7.04, 6.55, 6.17, 5.72, 5.38, 5.14, 4.96, 4.90, 4.84, 4.70, 4.59, 4.52, 4.33, 4.24, 4.15, 4.03, 3.91, 3.79, 3.70, 3.65, 3.54, 3.46, 3.36, 3.30, 3.20 and 3.11 (Å).

10. A crystalline salt of sunitinib with sulfuric acid.

11. A crystalline salt of sunitinib with sulfuric acid having an XRPD pattern comprising any five of interplanar spacing (d) values selected from the group consisting of 15.34, 13.23, 10.08, 9.53, 8.76, 7.66, 7.48, 7.04, 6.62, 6.54, 6.17, 6.06, 5.74, 5.49, 5.27, 5.10, 4.88, 4.82, 4.72, 4.50, 4.41, 4.31, 4.19, 4.07, 4.00, 3.97, 3.93, 3.83, 3.70, 3.65, 3.60, 3.54, 3.47, 3.44, 3.40, 3.27, 3.19 and 2.92 (Å).

12. A crystalline salt of sunitinib with acetic acid.

13. A crystalline salt of sunitinib with acetic acid having an XRPD pattern comprising any five of interplanar spacing (d) values selected from the group consisting of 23.47, 14.60, 12.35, 11.68, 9.74, 8.06, 7.79, 6.70, 6.53, 6.09, 5.59, 5.41, 5.16, 4.94, 4.86, 4.68, 4.40, 4.15, 4.04, 4.00, 3.80, 3.65, 3.60, 3.54, 3.47, 3.41, 3.32, 3.28 and 3.04 (Å).

14. A crystalline salt of sunitinib with methanesulfonic acid.

15. A crystalline salt of sunitinib with methanesulfonic acid having an XRPD pattern comprising any five of interplanar spacing (d) values selected from the group consisting of 15.18, 9.46, 8.58, 7.57, 7.03, 6.61, 6.36, 6.18, 5.78, 5.53, 5.28, 5.10, 5.04, 4.77, 4.72, 4.58, 4.34, 4.28, 4.21, 4.06, 3.91, 3.83, 3.78, 3.71, 3.68, 3.64, 3.58, 3.51, 3.43, 3.35, 3.26, 3.23, 3.15 and 3.00 (Å).

16. A process for the preparation of a salt of sunitinib with sulfuric acid, acetic acid, methanesulfonic acid, ethanesulfonic acid or succinic acid-, wherein the process comprises treating sunitinib with sulfonic acid, acetic acid, methanesulfonic acid, ethanesulfonic acid or succinic acid.

17. A process according to the claim 16, wherein the treatment with the achiral acid is carried out in the presence of a solvent.

18. A process according to the claim 17, wherein the solvent comprises organic solvents.

19. A process according to the claim 18, wherein the organic solvent comprises a water-miscible organic solvent.

20. A process according to the claim 19, wherein the water-miscible organic solvent comprises a C₁₋₃alkanol.

21. (canceled)
